(19) United States
(12) Patent Application Publication
(10) Pub. No.: US 2023/0099811 A1
(43) Pub. Date: Mar. 30, 2023

(54) COMPOSITION OF HYALURONIC ACID AND USE ALONE AND IN COMBINATION WITH RETINOIDS TO IMPROVE SKIN

(71) Applicant: RoC OPCO LLC, New York, NY (US)

(72) Inventors: Arthur Pellegrino, Newtown, PA (US); Ellen Delisle, Hauppauge, NY (US); Lea Benkoski, New York, NY (US); Fred Khoury, Chatsworth, CA (US)

(21) Appl. No.: 17/887,129

(22) Filed: Aug. 12, 2022

Related U.S. Application Data
(60) Provisional application No. 63/232,561, filed on Aug. 12, 2021.

(51) Int. Cl. A61K 8/73; A61K 8/67; A61Q 19/00

(52) U.S. Cl. CPC A61K 8/735; A61K 8/73; A61K 8/671; A61Q 19/00; A61K 2800/31

(57) ABSTRACT

The present invention relates to methods of increasing one or more of collagen, elastin, fibronectin, and hyaluronic acid gene expression in the skin of a subject comprising topically administering to the subject a hyaluronic acid compound and a retinoid in one or more compositions. The methods improve the look, feel and/or appearance of the skin, hydrate the skin, plump the skin, improve skin elasticity, improve the appearance of fine lines and wrinkles, reduce the depth, length, or width of fine lines and wrinkles, and smooth the skin. The present invention also relates to cosmetic compositions comprising a hyaluronic acid compound wherein the composition is anhydrous.

### CROSS REFERENCE TO RELATED APPLICATIONS

This application claims priority to U.S. Provisional Application 63/232,561 filed Aug. 12, 2021, the entire contents of which is incorporated by reference.

### FIELD OF INVENTION

Hyaluronic acid and retinoids have been extensively studied and utilized in skin care in the last several decades. However, despite their widespread use, little is known about their effects individually or in combination on hyaluronic acid replenishment and/or stimulation of production in the skin, and the corresponding impact on hydration and anti-aging properties such as reduction of fine lines and wrinkles, and improvements in firming and plumping within the skin. The present invention is directed to improved cosmetic formulations and treatment methods that harness and deliver these unexplored beneficial effects of hyaluronic acid and retinoids, such as retinol, to the skin.

### SUMMARY

One aspect of the invention includes a cosmetic composition of hyaluronic acid, wherein the composition is anhydrous.

Another aspect of the invention includes a method of improving skin by topically administering a hyaluronic acid (HA) compound and a retinoid in one or more compositions to the skin of a subject, wherein improving the skin includes (a) increasing one or more of collagen, elastin, fibronectin, and hyaluronic acid gene expression in the skin of a subject and/or (b) improving the look, feel and/or appearance of the skin, by hydrating the skin; plumping the skin; improving skin elasticity; improving the appearance of fine lines and wrinkles; reducing the depth, length, or width of fine lines and wrinkles; and/or smoothing the skin; and/or (c) increasing and/or replenishes the level of hyaluronic acid on the surface of the skin and/or in the skin; wherein the HA compound is hyaluronic acid or a salt thereof.

Another aspect of the invention includes a method of improving skin by administering the cosmetic composition to improve the look, feel and/or appearance of the skin.

Another aspect of the invention includes a method of improving skin by topically administering to the skin hyaluronic acid and a retinoid to improve the look, feel and/or appearance of the skin.

### DETAILED DESCRIPTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

Unless otherwise indicated, all parts and percentages are by weight. As used herein, the term “about” refers to plus or minus 10% of the indicated value. Unless otherwise stated or made clear by context, weight percentages are provided based on the total amount of the composition in which they are described. As used herein, the singular forms “a,” “an,” and “the” include plural referents unless the context clearly dictates otherwise.

Described herein are methods of treating the skin of a subject comprising topically administering hyaluronic acid and a retinoid. Also described herein are cosmetic compositions comprising hyaluronic acid. Also described herein are cosmetic compositions comprising a retinoid, such as retinol. Also described herein are cosmetic compositions comprising hyaluronic acid and a retinoid in combination with one another.

Hyaluronic Acid

In this description, the terms “hyaluronic acid compound,” and “HA compound” are used interchangeably and refer to free hyaluronic acid, hyaluronic acid derivatives having similar activity to the free acid (for example crosslinked hyaluronic acids), and salts of the free acid or derivative thereof. Unless required otherwise by context, the terms “hyaluronic acid” and “HA” are used interchangeably to refer to one or both of the free acid or an acceptable salt thereof. Acceptable salts include, but are not limited to, alkali metal salts such as potassium hyaluronate and sodium hyaluronate. In exemplary embodiments, the HA is sodium hyaluronate.

According to the invention, the HA compound can come from any source, including, but not limited to, synthetic sources and natural sources. Traditionally, HA was extracted from rooster combs. However, this traditional method faces growing concern over the use of animal-derived components in biomedical, pharmaceutical, and cosmetic applications. HA can also be produced via streptococcal fermentation which has lower production costs, produces less environmental pollution, and produces a mixture of HA of different molecular weights. Other recent HA production processes include recombinant production from gram-positive and gram-negative bacteria, such as Bacillus sp., Lactococcos lactis, Agrobacterium sp., and Escherichia coli. Further, recombinant microorganisms like Bacilli and Escherichia coli are endotoxin-free, making them a safer, alternative HA production source.

According to the invention, one or more HA compound having a particular molecular weight can be used in the methods and compositions of the present invention and can be linear, crosslinked, or crosslinked with other components. Typically, HA has a molecular weight of from about 1 kDa to about 20,000 kDa. In an embodiment of the invention, the HA compound has a molecular weight of about 1 kDa to about 20,000 kDa, for example, about 1 kDa to about 10,000, about 1 kDa to about 5,000 kDa, about 1 kDa to about 1,000 kDa, about 1 kDa to about 100 kDa, about 25 kDa to about 100 kDa, about 25 kDa to about 50 kDa, or about 50 kDa or less. In other embodiments of the invention, the HA compound has a molecular weight of about 10 kDa to about 40 kDa, for example, about 1 kDa to about 20 kDa, or about 3 kDa to about 10 kDa. In another embodiment of the invention, the HA compound has a molecular weight of about 500 kDa to about 2000 kDa, for example about 600 kDa to about 1000 kDa. In a preferred embodiment, the HA compound has a molecular weight of 10 kDa to 40 kDa. HAs with different molecular weights can obtained by selective production methods that generate particular molecular weight ranges or by controlled hydrolysis of higher molecular weight HAs.

HA Compositions

HA compositions of the present invention can be formulated as creams, gels, lotions, emulsions, or serums, any of which can be anhydrous, aqueous, oil-in-water, or water-in-oil formulations. Accordingly, according to some embodiments, the HA composition can include additional cosmetic ingredients or excipients including preservatives, humectants, surfactants and other polymers.

Preferably the HA composition is anhydrous. Most preferably, the anhydrous composition is stored in a single-use capsule to maintain the anhydrous nature of the formulation.

HA is well known to absorb large amounts of water and expand up to 1000 times in volume upon contact with sufficient water. The anhydrous nature of the formulations disclosed herein prevents the HA compound from activating and retaining water until it meets the skin. By applying an anhydrous formulation, the HA compound better penetrates into the skin since it has not yet had time to activate and expand. Once in the skin, the HA compound molecules in the anhydrous formulation activate and pull water into the skin both from the air and from within deeper layers of the skin itself. This also allows for administration of a significantly lower amount of HA compound as compared to known aqueous HA formulations, but while still achieving comparable and/or increased efficacy of the methods disclosed herein.

In embodiments of the invention, the HA compositions contain about 0.001 wt. % to about 3.00 wt. % HA compound. In embodiments of the invention, the HA compositions contain about 0.001 wt. % HA compound, about 0.005 wt. % HA compound, about 0.010 wt. % HA compound, about 0.015 wt. % HA compound, about 0.2 wt. % HA compound, about 0.3 wt. % HA compound, about 0.4 wt. % HA compound, about 0.5 wt. % HA compound, about 0.6 wt. % HA compound, about 0.7 wt. % HA compound, about 0.8 wt. % HA compound, about 0.9 wt. % HA compound, about 1.0 wt. % HA compound, about 1.5 wt. % HA compound, about 2.0 wt. % HA compound, about 2.5 wt. % HA compound, or about 3.0 wt. % HA compound. In an exemplary embodiment, the HA compositions contain about 0.006 wt. % HA compound.

Retinoids

Retinoids are a class of chemical compounds that are derivatives and analogs of vitamin A, and are used in cosmetics and medicines to regulate epithelial cell growth to treat photoaging and skin wrinkles. Four generations of retinoids exist.

First generation retinoids include retinol, retinal, tretinoin (i.e. retinoic acid or retin-A), isotretinoin, and alitretinoin.

Second generation retinoids include etretinate and its metabolite acitretin.

Third generation retinoids include adapalene, bexarotene, and tazarotene.

Fourth generation retinoids include trifarotene.

Retinol is the retinoid most frequently recommended by dermatologists for slowing the signs of skin aging and helping to maintain a youthful appearance. Retinol is a form of Vitamin A that naturally occurs in human skin. When employed regularly in a topical treatment, it can help improve skin firmness, visibly reduce the appearance of fine lines and deep wrinkles—and even help minimize the look of crow's feet and dark circles in the eye area.

Any retinoid can be used in the disclosed methods and compositions, with retinol being preferred.

According to the invention, the retinoid can be administered in a separate retinoid composition or is present in the HA composition (i.e. as a combination composition).

In embodiments of the invention, the retinol composition of the invention (i.e. a retinol composition to practice methods of the invention in combination with a separate hyaluronic acid composition or as part of a combination composition of the invention) contains about 0.001 wt. % to about 5.0 wt. % of retinoid. In embodiments of the invention, the composition contains about 0.001 wt. % retinoid, about 0.005 wt. % retinoid, about 0.010 wt. % retinoid, about 0.015 wt. % retinoid, about 0.2 wt. % retinoid, about 0.3 wt. % retinoid, about 0.4 wt. % retinoid, about 0.5 wt. % retinoid, about 0.6 wt. % retinoid, about 0.7 wt. % retinoid, about 0.8 wt. % retinoid, about 0.9 wt. % retinoid, about 1.0 wt. % retinoid, about 1.5 wt. % retinoid, about 2.0 wt. % retinoid, about 2.5 wt. % retinoid, about 3.0 wt. % retinoid, about 3.5 wt. % retinoid, about 4.0 wt. % retinoid, about 4.5 wt. % retinoid, or about 5.0 wt. % retinoid. Preferably, the composition contains about 0.15 wt. % or about 0.20 wt. %. Preferably, the retinoid is retinol.

An exemplary commercial source of retinoid is Retinol 10S (BASF), which contains 10.0 wt. % retinol and 0.1 wt. % BHT in soybean oil. Other sources of retinoids can be used with the invention.

Retinoid Compositions

Retinoid compositions of the present invention can be formulated as creams, gels, lotions, or serums, any of which can be anhydrous, aqueous, oil-in-water, or water-in-oil formulations.

Preferably the retinoid composition is an anhydrous serum composition. Most preferably, the anhydrous serum composition is stored in a single-use capsule to maintain the anhydrous nature of the formulation. Further, by storing the retinol serum in a single-use capsule, the serum is sealed off from the air, thereby preventing oxidation of the retinol to maintain its efficacy.

Konjac Glucomannan

Konjac glucomannan is a vegetal polysaccharide with high molecular weight, typically in the range of 200 kDa to 2000 kDa, and has exceptional hygroscopic properties which allow it to absorb up to 200 times its weight in water. It comes from the root powder of Amorphophallus konjac, which is a plant of the Araceae family that grows naturally in the forests of Thailand, Vietnam, and southern China. In traditional Chinese medicine and Asian herbology, konjac is known as “devil's tongue” for its detoxifying and soothing properties.

In exemplary embodiments, the HA compound, for example free hyaluronic acid or a salt thereof, is crosslinked with konjac glucomannan in the form of dehydrated Ultra Filling Spheres™ (from BASF), as disclosed in US2015/0283055A1, the entire contents of which are incorporated by reference. Ultra Filling Spheres™ have been shown to penetrate the epidermis, absorbing water to smooth fine lines and deep wrinkles, improve elasticity, and provide long term hydration.

Ultra Filling Spheres™ contain HA as a combination of about 97.5 wt. % HA and about 2.5 wt. % konjac glucomannan, dispersed at a concentration of about 0.2 wt. % in ethylhexyl palmitate (about 97.8 wt. % of the total Ultra Filling Spheres™ formulation) and trihydroxystearin (about 2.0 wt. % of the total Ultra Filling Spheres™ formulation). The sodium hyaluronate present in the Ultra Filling Spheres™ typically has a molecular weight of 10 kDa to 40 kDa.

In embodiments of the invention, the HA compositions of the present invention contain about 1.0 wt. % to about 10.0 wt. % Ultra Filling Spheres™. In embodiments of the invention, the HA compositions contain about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, or about 10.0 wt. % Ultra Filling Spheres™. In a preferred embodiment, the HA compositions contain about 3.0 wt. % Ultra Filling Spheres™.

While one can use Ultra Filling Spheres™, other HA microspheres are suitable for use in the methods and compositions according to the invention. Alternative HA microspheres include, but are not limited to Hyaluronic Filling Spheres® also supplied by BASF, which use a hygroscopic material to make a sphere, but do not include konjac glucomannan. Other Hyaluronic Filling Spheres technology is based on the use of cross-linked spheres.

Methods

Hyaluronic acid is an anionic, non-sulfated glycosaminoglycan distributed widely throughout connective, epithelial, and neural tissues, and is a major component of the skin. As skin ages, the most dramatic change in the chemical constituents of the skin is the marked disappearance of epidermal HA, which is the principle molecule responsible for binding and retaining water molecules. This leads to a significant loss in the amount of moisture in the skin. Although HA remains in the dermis, as one ages there is a progressive reduction in the size of the HA polymers in the skin. Further still, as one ages there is a marked loss in extractability of HA into the extracellular space and thus an increasing need for increasing the amount of HA within tissue structures. This parallels the progressive cross-linking of collagen and a steady loss of collagen extractability with age. The combination of the loss of HA and collagen results in skin dehydration, atrophy, and decreased skin elasticity, leading to the tell-tale signs of aging, including fine lines and wrinkles.

HA is synthesized by specific enzymes called hyaluronic synthase, or HAS, and three different mammalian HAS enzymes exist: HAS1, HAS2, and HAS3. They are membrane bound enzymes and they synthesize HA on the inner surface of the plasma membrane, after which the HA is extruded through pore-like structures into the extracellular space. Studies assessing photo-protected skin tissues specimens from adults and juvenile patients have shown that intrinsic skin aging is associated with a significant reduction in the content of not only HA, but also downregulation of HAS-1 and HAS-2.

In order to effectively combat the signs of aging of the skin, one strategy is to first turn on the genes responsible for the expression of HAS in order to encourage the skin produce HA of higher molecular weight and facilitate its extrusion to the extracellular matrix of the skin.

According to embodiments of the invention, the methods disclosed herein involve topical administration of a hyaluronic acid compound alone or in combination with a retinoid to the skin of a subject to improve the skin, wherein the improvement achieves one or more of the following benefits:

i. Increasing collagen gene expression. The increases in collagen gene expression can be selected from one or more of collagen type-I (COL1A1), collagen type-III (COL3A1), and collagen type-IV (COL4A1).

ii. Increasing elastin (i.e. ELN) gene expression.

iii. Increasing fibronectin (i.e. FN1) gene expression.

iv. Increasing hyaluronic acid gene expression. The increases in hyaluronic acid gene expression can be selected from one or more of hyaluronic acid synthase 1 (i.e. HAS1) and hyaluronic acid synthase 2 (i.e. HAS2).

By upregulating these specific gene expressions in the skin, in other embodiments of the invention, one or more of the following benefits in the skin are also achieved by topical administration of hyaluronic acid alone or in combination with a retinoid to the skin of a subject:

i. Increasing hydration in the skin.

ii. Increasing and replenishing the level of hyaluronic acid on the surface of the skin.

iii. Increasing and replenishing the level of hyaluronic acid in the skin.

In other embodiments of the invention, topical administration of hyaluronic acid in combination with a retinoid results in one or more of the following improvements in the look, feel, and appearance of the skin:

i. Increasing skin firmness. Increases in skin firmness include visibly boosting the appearance/look of firmer skin by 1.5×. This visible boost in the appearance/look of firmer skin can include a 1.5× boost in collagen support in the skin.

ii. Increasing skin plumpness. Increases in skin plumpness include visibly boosting the appearance/look of plumper, fuller skin by 7×. This visible boost in appearance of the plumper, fuller skin can include a 7× boost in hyaluronic acid levels in the skin.

iii. Improving fine lines and wrinkles of the skin. Improvements in fine lines and wrinkles of the skin include reducing the length, depth, and/or width of fine lines and/or wrinkles and/or reducing the appearance of fine lines and/or wrinkles.

iv. Increasing smoothness of the skin.

v. Increasing elasticity of the skin.

According to the invention, the hyaluronic acid and the retinoid can be administered in one or more compositions. The administration can be in separate compositions, i.e. a HA composition and a retinoid composition, or in one composition (a combination composition), to administer the hyaluronic acid and the retinoid simultaneously. When administered in two separate compositions, the hyaluronic acid composition can be administered before the retinoid composition or after the retinoid composition. When administered in two separate compositions, the hyaluronic acid composition is preferably administered before the retinoid composition, and the retinoid composition is preferably administered immediately after the hyaluronic acid composition is administered to the skin. Preferably the retinoid composition is a retinol composition.

According to the invention, the method includes administration at any time of the day, including the morning, afternoon, and evening. In some embodiments of the invention, the HA and retinoid compositions or a combination composition are administered in the morning and/or the evening. In an exemplary embodiment, the HA and retinoid compositions or a combination composition is administered in the evening, such as at the subject's bedtime. In other exemplary embodiments one of the HA or retinoid compositions is administered in the morning, and the other of the retinoid or HA compositions is administered in the evening.

In some embodiments of the method, the composition(s) to be administered is contained in a capsule. In such compositions, the method includes the following steps:

1) Twisting open the capsule(s);

2) Administering (or applying) the hyaluronic acid composition from the capsule to the skin;

3) Administering (or applying) retinol composition from the capsule to the skin.

Alternatively, the hyaluronic acid composition from the hyaluronic acid capsule and the retinol composition from the retinol capsule can be administered simultaneously, for example, by mixing together in the palm of the subject, and applying together simultaneously, or by applying the retinol composition immediately upon applying the HA composition.

In embodiments, in initial administrations, the subject applies the composition(s) every other night until the skin has become accustomed to the products. Administration frequency can be increased as necessary, preferably until able to administer the composition(s) every evening/night. If applying the composition(s) during the day, the subject can also apply a sunscreen of SPF15 or higher after applying the retinol containing composition.

According to the invention, the skin to be treated can be cleansed prior to administration of the HA and retinoid compositions or the combination composition.

According to the invention skin in any area can be treated. In exemplary embodiments of the invention, the skin to be treated is the skin of the face, of the neck, or of both the face and the neck. In other exemplary embodiments of the invention, the skin is the skin of the chest/decolletage.

Other Components

Coconut Alkanes

Coconut alkanes are a natural product obtained from the complete reduction and hydrogenation of a mixture of fatty acids derived from coconut oil, and function as an emollient in skin & hair care applications.

In embodiments of the invention, the HA compositions contain about 30.0 wt. % to about 45.0 wt. % coconut alkanes. In a preferred embodiment, the HA compositions contain about 37.4 wt. % coconut alkanes.

Coco-Caprylate/Caprate

Coco-caprylate/caprate is a vegetable ingredient obtained from coconut with a high level of biocompatibility with the skin. It has the ability to penetrate deep and help skin to repair itself, acting as an emollient and providing the skin with softness.

In embodiments of the invention, the HA compositions contain about 1.0 wt. % to about 5.0 wt. % coco-caprylate/caprate. In a preferred embodiment, the HA compositions contain about 2.4 wt. % coco-caprylate/caprate.

An exemplary commercial source of coco-caprylate/caprate is Vegelight 1214LC, which is a volatile and lower odor alkane sourced from vegetable oils. It is a clear, colorless emollient with volatility properties similar to petroleum-derived isododecane and synthetically-derived cyclomethicones. It is ideal for application in skincare, color cosmetic, sun care, deodorant, and hair care formulations. Vegelight 1214LC contains 6.0 wt. % coco-caprylate/caprate in coconut alkanes and is supplied by Grant Industries. Other sources of coco-caprylate/caprate are also acceptable.

Jojoba Seed Oil

Jojoba seed oil is an emollient, non-fragrant oil extracted from the seeds of a perennial shrub. Jojoba oil has been shown to enhance the skin's restorative properties. It is a rich source of numerous fatty acids and can also provide topical skin-soothing benefits.

In embodiments of the invention, the HA compositions of the present invention contain about 20.0 wt. % to about 25.0 wt. % jojoba seed oil. In a preferred embodiment, the HA compositions contain about 22.0 wt. % jojoba seed oil.

Ethylene/Propylene/Styrene Copolymer and Butylene/Ethylene/Propylene Copolymer

Ethylene/propylene/styrene copolymer is a helper ingredient that is used as an oil gelling agent together with the related compound butylene/ethylene/styrene copolymer. These two together can be combined with different types of hydrocarbons (e.g. mineral oil or different emollient esters) to form gels with different sensorial and physical properties. The resulting hydrocarbon gels can improve skin occlusivity, reduce trans-epidermal water loss, and are also able to form suspensions.

In embodiments of the invention, the HA compositions of the present invention contain about 1.0 wt. % to about 5.0 wt. % ethylene/propylene/styrene copolymer. In a preferred embodiment, the HA compositions contain about 2.5 wt. % ethylene/propylene/styrene copolymer.

In embodiments of the invention, the HA compositions of the present invention contain about 0.1 wt. % to about 1.0 wt. % butylene/ethylene/styrene copolymer. In a preferred embodiment, the HA compositions contain about 0.5 wt. % butylene/ethylene/styrene copolymer.

An exemplary commercial source of ethylene/propylene/styrene copolymer in combination with butylene/ethylene/styrene copolymer is Jojoba Glaze HV BF, which is a clear high viscosity gel composed almost entirely of jojoba oil which provides gloss and shine to skin and hair while delivering substantive emolliency. It offers excellent suspension properties for glitters, sugar and other particles. Jojoba Glaze HV BF contains i) 9.9 wt. % ethylene/propylene/styrene copolymer, ii) 2.0 wt. % butylene/ethylene/propylene copolymer, and iii) 0.10 wt. % butylated hydroxytoluene (i.e. BHT) in jojoba seed oil, and is supplied by Vantage Personal Care. Other commercial sources of ethylene/propylene/styrene copolymer and butylene/ethylene/styrene are also acceptable.

Polyurethane-79

Polyurethane-79 is a copolymer formed by reacting hydrogenated polybutanediol, 1,6-hexamethylene diisocyanate, hydrogenated dilinoleyl alcohol, and 1,4-butanediol. The polymer is capped with stearyl alcohol. Polyurethane-79 is utilized in cosmetics as a film forming agent on the skin, hair and nails, and as viscosity modifier.

In embodiments of the invention, the HA compositions contain about 0.5 wt. % to about 2.5 wt. % polyurethane-79. In a preferred embodiment, the HA compositions contain about 1.2 wt. % polyurethane-79.

An exemplary commercial source of polyurethane-79 is Oilkemia 5S Polymer, which is an oil soluble rheology modifier that provides excellent thickening efficiency, clarity, suspension and stability, together with a pleasant non-tacky feel to create enchanting textures across skin care, sun care, and color cosmetic applications. An exemplary source of polyurethane-79 is Oilkemia 5S Polymer, which contains 30.0 wt. % polyurethane-79 in caprylic/capric triglyceride, and is supplied by Lubrizol. Other sources of polyurethane-79 are also acceptable.

Caprylic/Capric Triglyceride

Caprylic/capric triglyceride is a traditional, medium spreading emollient for modern cosmetic applications. It is a clear, slightly yellowish, polar, odorless oil with a spreading value of approximately 550 mm²/10 min. It has a refractive index (20° C.) of 1.448-1.450, a density (20° C.) of 0.943-0.950 g/ml, and a saponification value of 330-340. An exemplary commercial source of caprylic/capric triglyceride is Myritol 312, which is supplied by BASF. Other sources of caprylic/capric triglyceride are also acceptable.

In embodiments of the invention, the HA compositions contain about 15.0 wt. % to about 25.0 wt. % caprylic/capric triglyceride. In a preferred embodiment, the HA compositions contain about 19.0 wt. % caprylic/capric triglyceride.

Coconut Oil

Coconut oil is derived from the meat of the coconut. The oil is extracted, refined, bleached, and deodorized and has applications in the cosmetic industry as an ingredient in soaps, skin moisturizers, and tanning lotions, among others. An exemplary commercial source of coconut oil is Brenntag. Other sources of coconut oil are also acceptable.

In embodiments of the invention, the HA composition contains about 5.0 wt. % to about 15.0 wt. % coconut oil. In a preferred embodiment, the HA composition contains about 10.0 wt. % coconut oil.

Isosorbide Dicaprylate

Isosorbide dicaprylate is a natural, smart lipophilic hydrator and skin barrier builder. Stimulating Aquaporins-3, it provides long-lasting hydration (>48 hours) and contributes to maintain a healthy barrier function by up-regulating tight junctions, desmosomes and ceramide synthase. An exemplary commercial source of isosorbide dicaprylate is Hydra Synol DOI, which is supplied by Syntheon. Other sources of isosorbide dicarpylate are also acceptable.

In embodiments of the invention, the HA compositions contain about 1.0 wt. % to about 3.0 wt. % isosorbide dicaprylate. In a preferred embodiment, the HA compositions contain about 2.0 wt. % isosorbide dicaprylate.

Cyclopentasiloxane

Cyclopentasiloxane is a cyclic, volatile silicone fluid which can be used in a wide variety of skin, hair, and color personal care applications. It has excellent transient emolliency, and is one of the most widely used and important cosmetic solvents currently used. Cyclopentasiloxane is recommended for use in antiperspirants and deodorants, skin lotions, hair sprays, nail polishes, shaving lotions, perfumes and colognes, and make-up. It can also be used in air care evaporative applications such as diffusers.

In embodiments of the invention, the retinol composition of the invention (i.e. a retinol composition to practice methods of the invention in combination with a separate hyaluronic acid composition or as part of a combination composition of the invention) contains about 55.0 wt. % to about 70.0 wt. % cyclopentasiloxane. In a preferred embodiment, the retinol composition contains about 61.2 wt. % cyclopentasiloxane.

Dimethiconol

Dimethiconol, also referred to as silicone gum, is a polymer similar to dimethicone. It is a type of silicone that is used in skincare and hair care products. Dimethiconol, as a silicone, helps to improve the appearance, texture and feel of the product, prevent moisture loss from the skin, and enhance the efficacy of the product.

In embodiments of the invention, the retinol composition of the invention (i.e. a retinol composition to practice methods of the invention in combination with a separate hyaluronic acid composition or as part of a combination composition of the invention) contains about 5.0 wt. % to about 15.0 wt. % dimethiconol. In a preferred embodiment, the retinol composition contains about 10.8 wt. % dimethiconol.

An exemplary commercial source of dimethiconol is Xiameter PMX-1501 Fluid, which is a blend of an ultra-high viscosity dimethiconol in cyclopentasiloxane. This film forming, clear viscous fluid is long lasting and resistant to wash off making it suitable for durable cosmetics. In hair care products, it conditions hair, especially split ends. In skin care products, it imparts a soft, velvety skin feel. This product may be used in a wide range of cosmetic and toiletry applications such as skin care, color cosmetics, sun care, hair care, shower gels, antiperspirants and deodorants. Xiameter PMX-1501 Fluid contains 15.0 wt. % dimethiconol in cyclopentasiloxane, and is supplied by Univar Solutions. Other sources of dimethiconol and cyclopentasiloxane are also acceptable.

Ethylhexyl Cocoate

Ethylhexyl cocoate is an ester that can be used to limit the concentration of silicones in formulations and offers an alternative for surface feels that are perceived as more natural. It also produces a soft finish that is especially perceptible in facial and body care formulas.

In embodiments of the invention, the retinol composition of the invention (i.e. a retinol composition to practice methods of the invention in combination with a separate hyaluronic acid composition or as part of a combination composition of the invention) contains about 15.0 wt. % to about 25.0 wt. % ethylhexyl cocoate. In a preferred embodiment, the retinol composition contains about 20.0 wt. % ethylhexyl cocoate.

Soybean Oil

Soybean Oil (Glycine soja oil) is an oil which contains unsaturated fatty acids, soy lecithin, and also the essential alpha-linolenic acid with uses in cosmetics, food, and pharmceuticals. An exemplary commercial source of soybean oil is Refined Soybean Oil IP, which is supplied by Gustav Heess. Other sources of soybean oil are acceptable.

In embodiments of the invention, the retinol compositions contain about 1.0 wt. % to about 10.0 wt. % soybean oil. In a preferred embodiment, the retinol compositions contain about 7.0 wt. % soybean oil.

Phenoxyethanol

Phenoxyethanol is an antimicrobial preservative. It is widely used as an antimicrobial preservative for cosmetic, toiletry and pharmaceutical applications, such as shampoos, foam baths, shower gels or liquid detergents. This product is chemically inert and is therefore compatible with the majority of types of chemical compounds. An exemplary commercial source of phenoxyethanol is Phenoxetol, which is supplied by Clariant. Other sources of phenoxyethanol are acceptable.

In embodiments of the invention, the retinol composition of the invention (i.e. a retinol composition to practice methods of the invention in combination with a separate hyaluronic acid composition or as part of a combination composition of the invention) contains about 0.5 wt. % to about 1.5 wt. % phenoxyethanol. In a preferred embodiment, the retinol composition contains about 0.8 wt. % phenoxyethanol.

Dimethylmethoxy Chromanol

Dimethylmethoxy chromanol is an antioxidant, analog to γ-tocopherol, that confers triple protection from reactive species (ROS, RNS, RCS) and may aid in detoxification. When applied to the skin, antioxidative properties and parameters associated with aging were improved, while a depigmenting activity was also measured. An exemplary commercial source of dimethylmethoxy chromanol is Lipochroman molecule, which is supplied by Lipotec. Other sources of dimethylmethoxy chromanol are acceptable.

In embodiments of the invention, the retinol composition of the invention (i.e. a retinol composition to practice methods of the invention in combination with a separate hyaluronic acid composition or as part of a combination composition of the invention) contains about 0.005 wt. % to about 0.015 wt. % dimethylmethoxy chromanol. In a preferred embodiment, the retinol composition contains about 0.01 wt. % dimethylmethoxy chromanol.

Ceramide III

Ceramide III is a ceramide that reinforces the skins natural protective lipid barrier. Ceramide III consists of a Phytosphingosine backbone acylated with a saturated fatty acid (stearic acid). Ceramide III and Ceramide IIIB support the renewal of the skin's natural protective layer and form an effective barrier against moisture loss. These human-skin-identical molecules are therefore particularly suitable for long term protection and repair of sensitive and dry skin. In hair care formulations Ceramide III and Ceramide IIIB are able to restore damaged hair and to protect hair against chemical and UV damage. An exemplary commercial source of Ceramide III is Evonik. Other sources of Ceramide III are acceptable.

In embodiments of the invention, the retinol composition of the invention (i.e. a retinol composition to practice methods of the invention in combination with a separate hyaluronic acid composition or as part of a combination composition of the invention) contains about 0.00005 wt. % to about 0.00015 wt. % Ceramide III. In a preferred embodiment, the retinol composition contains about 0.0001 wt. % Ceramide III.

Exemplary Compositions

Exemplary HA compositions according to the invention are disclosed in Table A below:

TABLE A
HA Compositions

| Ingredient | Amount (wt. %) |
|---|---|
| Coconut Alkanes | 30.0-45.0 |
| Coco-Caprylate/Caprate | 1.0-5.0 |
| Simmondsia Chinensis | 20.0-25.0 |
| (Jojoba) Seed Oil | |
| Ethylene/Propylene/ | 1.0-5.0 |
| Styrene Copolymer | |
| Butylene/Ethylene/ | 0.1-1.0 |
| Propylene Copolymer | |
| Caprylic/Capric | 15.0-25.0 |
| Triglyceride | |
| Cocos Nucifera | 5.0-15.0 |
| (Coconut) Oil | |
| Polyurethane-79 | 0.5-2.5 |
| Hyaluronic Acid | 0.001-3.00 |
| Konjac glucomannan | 0-0.001 |
| Isosorbide Dicaprylate | 1.0-3.0 |

A specific example of an HA composition according to the invention is disclosed in Table B below:

TABLE B
Exemplary Hyaluronic Acid Composition

| | Individual |
|---|---|
| | Ingredient |
| | amount |
| Individual Ingredients | (wt. %) |
| Coconut Alkanes | 37.365 |
| Coco-Caprylate/Caprate | 2.385 |
| Simmondsia Chinensis | 22.000 |
| (Jojoba) Seed Oil | |
| Ethylene/Propylene/Styrene | 2.475 |
| Copolymer | |
| Butylene/Ethylene/Propylene | 0.500 |
| Copolymer | |
| BHT | 0.025 |
| Caprylic/Capric Triglyceride | 16.250 |
| Cocos Nucifera (Coconut) | 10.000 |
| Oil | |
| Caprylic/Capric Triglyceride | 2.800 |
| Polyurethane-79 | 1.200 |
| Ethylhexyl Palmitate | 2.934 |
| Trihydroxystearin | 0.060 |
| Sodium Hyaluronate | 0.006 |
| Konjac glucomannan | 0.00015 |
| Isosorbide Dicaprylate | 2.000 |

In the above composition of Table B, the ingredients can be provided as follows: Coconut Alkanes and Coco-Caprylate/Caprate may be provided by Vegelight 1214LC; Simmondsia Chinensis (Jojoba) Seed Oil, Ethylene/Propylene/Styrene Copolymer, Butylene/Ethylene/Propylene Copolymer, and BHT may be provided as Jojoba Glaze HV; Caprylic/Capric Triglyceride may be provided as Myritol 312; Cocos Nucifera (Coconut) Oil may be provided as Olio DI COCCO Raffinate; Caprylic/Capric Triglyceride and Polyurethane-79 may be provided as Oilkemia 5S Polymer; Ethylhexyl Palmitate, Trihydroxystearin, Sodium Hyaluronate, and Konjac glucomannan may be provided as Ultra Filling Spheres; and Isosorbide Dicaprylate may be provided as HydraSynol DOI.

Anhydrous HA serum capsules containing 0.006 wt. % HA compound disclosed herein were tested against a commercially available aqueous HA serum formulation containing 1.5 wt. % HA (Example 2 below). Despite the 1.5 wt. % HA aqueous formulation containing 25,000% more HA, the anhydrous capsules achieved significantly better results than the aqueous formulation when HA levels were measured in both culture medium and tissue lysates. Additionally, the anhydrous HA formulation successfully induced expression of HAS1 and FN1, which was not observed with the aqueous HA formulation. When combined with a retinoid, the anhydrous HA serum unexpectedly performed even better.

Notably, when the HA anhydrous formulation was combined with retinol, a significant increase was seen in COL1A1 (48%), COL3A1 (58%), and COL4A1 (79%) which was not observed for either product alone, nor the comparative 1.5 wt. % HA aqueous formulation or commercially available 0.3 wt. % aqueous retinol formulation. In fact, in the case of COL4A1, both aqueous formulations significantly decreased gene expression. Further, the expression levels of ELN and FN1 were notably higher for the combinations according to the invention, despite the HA and retinol concentrations being significantly lower in their corresponding aqueous formulations.

While a 298% and 233% increase were observed for HAS2 with the individual aqueous formulations, the combination according to the invention delivered an unexpected 638% increase in HAS2 expression levels after 24 hours. It is also significant to note that the aqueous 0.3 wt. % retinol serum tested also contains hyaluronic acid present as sodium hyaluronate at even higher levels than the retinol. Therefore, despite the aqueous retinol product also containing a HA/retinol combination, it only increased expression levels by a third of the increase observed for the combination of the invention.

In an embodiment, the HA composition includes HA compound crosslinked with konjac glucomannan, and one or more additional components selected from coconut alkanes, coco-caprylate/caprate, jojoba seed oil, ethylene/propylene/styrene copolymer and butylene/ethylene/propylene copolymer, BHT, caprylic/capric triglyceride, coconut oil, polyurethane-79, ethylhexyl palmitate, trihydroxystearin, and isosorbide dicaprylate.

In any of the above compositions, the HA compound crosslinked with konjac glucomannan, coconut alkanes, coco-caprylate/caprate, jojoba seed oil, ethylene/propylene/styrene copolymer and butylene/ethylene/propylene copolymer, BHT, caprylic/capric triglyceride, coconut oil, polyurethane-79, ethylhexyl palmitate, trihydroxystearin, and isosorbide dicaprylatecan be provided by using the commercially available products Ultra Filling Spheres™, Vegelight 1214LC, Jojoba Glaze HV BF, Myritol 312, coconut oil, Oilkemia 5S Polymer, and HydraSynol DOI.

Retinoid compositions according to the invention are disclosed in Table C.

TABLE C
Retinoid Compositions

| Ingredient | Amount (wt. %) |
|---|---|
| Cyclopentasiloxane | 55.0-70.0 |
| Dimethiconol | 5.0-15.0 |
| Ethylhexyl Cocoate | 15.0-25.0 |
| Glycine Soja | 1.0-10.0 |
| (Soybean) Oil | |
| Retinoid | 0.001-5.0 |
| Phenoxyethanol | 0.5-1.5 |
| Dimethylmethoxy | 0.005-0.15 |
| Chromanol | |
| Ceramide NP | 0.00005-0.00015 |

A specific example of a retinoid composition according to the invention is disclosed in Table D below:

TABLE D
Exemplary Retinoid composition

| | Individual |
|---|---|
| | Ingredient |
| | amount |
| Individual Ingredients | (wt. %) |
| Cyclopentasiloxane | 61.1914 |
| Dimethiconol | 10.7985 |
| Ethylhexyl Cocoate | 20.0000 |
| Glycine Soja (Soybean) Oil | 5.0000 |
| Glycine Soja (Soybean) Oil | 1.9778 |
| Retinol | 0.2200 |
| BHT | 0.0022 |
| Phenoxyethanol | 0.8000 |
| Dimethylmethoxy Chromanol | 0.0100 |
| Ceramide NP | 0.0001 |

In the above composition of Table D, the ingredients can be provided as follows: Cyclopentasiloxane and dimethiconol may be provided as Xiameter PMX-1501 Fluid; Ethylhexyl cocoate may be provided from any suitable commercial source; Glycine Soja may be provided as Refined Soybean Oil IP; Glycine Soja oil, retinol, and BHT may be provided as Retinol 10S; Phenoxyethanol may be provided as Phenoxetol; Dimethylmethoxy Chromanol may be provided as Liopchroman Molecule; and Ceramide NP may be provided as Ceramide III.

In an embodiment, the retinoid composition includes retinol and one or more additional components selected from cyclopentasiloxane, dimethiconol, ethylhexyl cocoate, soybean oil, phenoxyethanol, dimethylmethoxy chromanol, and ceramide.

In any of the above compositions, the retinol, cyclopentasiloxane, dimethiconol, ethylhexyl cocoate, soybean oil, phenoxyethanol, dimethylmethoxy chromanol, and ceramide can be provided using the commercially available products Retinol 10S, Xiameter PMX-1501 Fluid, Ethylhexyl cocoate, Refined Soybean Oil IP, Phenoxetol, Lipochroman molecule, and Ceramide III.

Combination Composition

As described above, advantages of the present invention can be obtained by application of the HA and retinoid in separate compositions or in a single combination HA/retinoid composition. Combination HA/retinoid compositions can be formulated as creams, gels, lotions, or serums, any of which can be anhydrous, aqueous, oil-in-water, or water-in-oil formulations. Preferably the combination HA/retinoid composition is an anhydrous serum composition. Most preferably, the anhydrous serum composition is stored in a single-use capsule to maintain the anhydrous nature of the formulation. By combining the HA compound and retinoid into a single anhydrous formulation that is stored in a sealed, single-use capsule, one can prevent activation of the HA compound prior to administration to the skin while simultaneously preventing oxidation and degradation of the retinoid. This ideal formulation and storage strategy can significantly increase the efficacy of the product and methods of the present invention, as well as product stability.

In an embodiment, the combination composition includes HA compound crosslinked with konjac glucomannan, a retinoid, and one or more additional components selected from coconut alkanes, coco-caprylate/caprate, jojoba seed oil, ethylene/propylene/styrene copolymer and butylene/ethylene/propylene copolymer, BHT, caprylic/capric triglyceride, coconut oil, polyurethane-79, ethylhexyl palmitate, trihydroxystearin, and isosorbide dicaprylate. In a preferred embodiment, the retinoid is retinol.

In any of the above compositions, the HA compound crosslinked with konjac glucomannan, the retinoid, coconut alkanes, coco-caprylate/caprate, jojoba seed oil, ethylene/propylene/styrene copolymer and butylene/ethylene/propylene copolymer, BHT, caprylic/capric triglyceride, coconut oil, polyurethane-79, ethylhexyl palmitate, trihydroxystearin, and isosorbide dicaprylatecan be provided by using the commercially available products Ultra Filling Spheres™, Retinol 10S, Vegelight 1214LC, Jojoba Glaze HV BF, Myritol 312, coconut oil, Oilkemia 5S Polymer, and HydraSynol DOI.

.Chromenone

Certain chromenone derivatives have been shown to exhibit anti-aging effects. U.S. Pat. No. 8,518,986, the entire contents of which are incorporated by reference, teaches chromenone derivatives of formula (I):

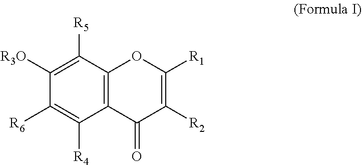

or a salt thereof where:

R¹ and R² are identical or different, and are selected from the group consisting of H, —(C═O)—R⁷, —C(═O)—OR⁷, a straight-chain or branched C₁- to C₂₀-alkyl group, wherein the alkyl is optionally at least once interrupted by oxygen, a straight-chain or branched C₃- to C₂₀-alkenyl group, a straight-chain or branched C₁- to C₂₀-hydroxyalkyl or -di- or polyhydroxyalkyl group, where the hydroxyl group is bonded to a primary or secondary carbon atom of the alkyl, and wherein the alkyl is optionally at least once interrupted by oxygen, a C₃- to C₁₀-cycloalkyl group and a C₃- to C₁₂-cycloalkenyl group (where the cyclic group is optionally bridged by —CH₂)ₙ— group where n=1 to 3);

R³ is H or a straight-chain or branched C₁- to C₂₀-alkyl group;

R⁴ is H or —OR⁸;

R⁵ and R⁶ are identical or different, and are selected from the group consisting of H or hydroxyl (OH), a straight-chain or branched C₁- to C₂₀-alkyl group (wherein the alkyl is optionally at least once interrupted by oxygen), a straight-chain or branched C₃- to C₂₀-alkenyl group, and a straight-chain or branched C₁- to C₂₀-hydroxyalkyl group, where the hydroxyl group is bonded to a primary or secondary carbon atom of the alkyl, and wherein the alkyl is optionally at least once interrupted by oxygen;

R⁷ is selected from the group consisting of H, a straight-chain or branched C₁- to C₂₀-alkyl group, wherein the alkyl is optionally at least once interrupted by oxygen, a straight-chain or branched C₃- to C₂₀-alkenyl group, and a straight-chain or branched C₁- to C₂₀-hydroxy-alkyl or -di- or polyhydroxyalkyl group, where the hydroxyl group is bonded to a primary or secondary carbon atom of the alkyl and wherein the alkyl is optionally at least once interrupted by oxygen, and

R⁸ is H or a straight-chain or branched C₁- to C₂₀-alkyl group.

Chromenones of formula (I) effectively potentiate the topical efficacy of retinoids. Preferred compounds of formula (I) for use in the methods, HA compositions, or retinoid compositions of the invention include compounds 1-11 shown below:

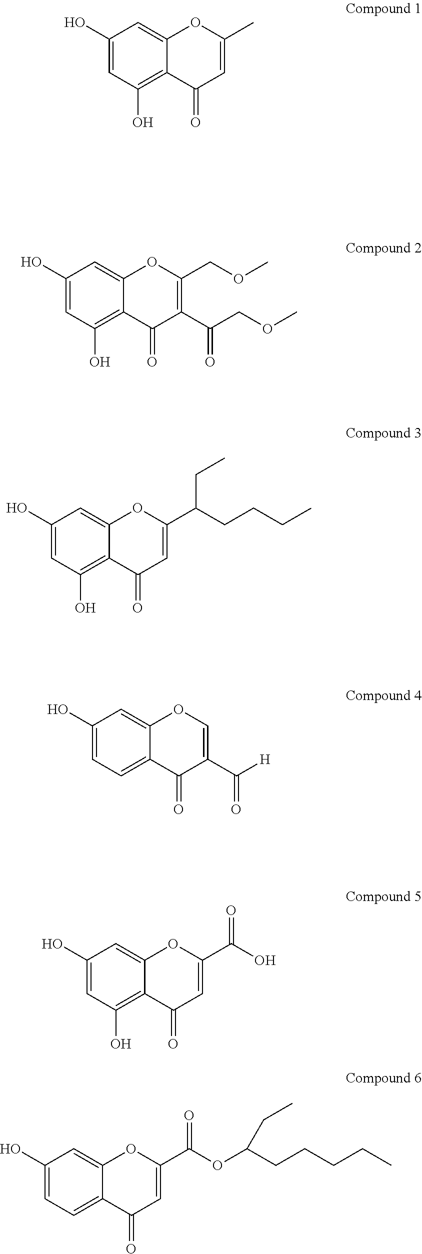 [SMILES: Cc1cc(=O)c2c(O)cc(O)cc2o1 ; COCC(=O)c1c(COC)oc2cc(O)cc(O)c2c1=O ; CCCCC(CC)c1cc(=O)c2c(O)cc(O)cc2o1 ; O=Cc1coc2cc(O)ccc2c1=O ; O=C(O)c1cc(=O)c2c(O)cc(O)cc2o1 ; CCCCCC(CC)OC(=O)c1cc(=O)c2ccc(O)cc2o1]

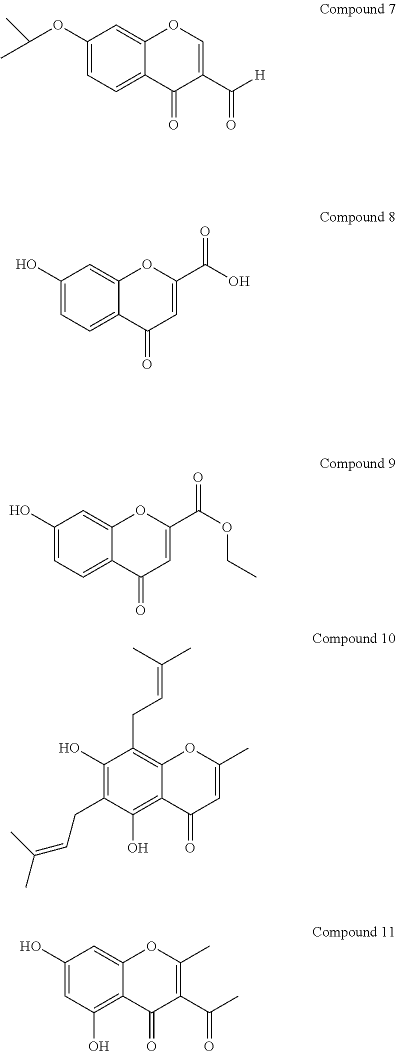

In certain embodiments, the HA or the retinoid compositions can contain about 0.1 wt. % to about 2 wt. % of at least one chromenone compound of formula (I). Preferably the HA composition only contains the chromenone compound when the HA compositions also contains a retinoid. Retinol is the preferred retinoid for use in combination with the compound of formula (I).

### EXAMPLES

Clinical Efficacy Study

The hyaluronic acid serum capsule formulation of Table B was utilized in the following clinical efficacy study.

The study was conducted with 33 female subjects to determine if the formulation improved global fine lines and wrinkles, skin moisture, skin firmness, skin elasticity and skin barrier function immediately after first use and after 1, 4 and 8 weeks product use, when used once a day.

### Example 1.1: Global Fine Lines and Wrinkles—Image Analysis

At baseline and after 1, 4 and 8 weeks of product use, a trained technician took digital images of the face of each subject. Using ImagePro® software, the images were analyzed to determine changes in the appearance of global wrinkles. A decrease in the score represents an improvement. An increase represented a worsening. Table 1.1 presents a summary of the fine lines and wrinkles analysis.

TABLE 1.1
Global Fine Lines and Wrinkles-Image Analysis

| | | | Mean | |
|---|---|---|---|---|
| | | | Change | % of Subjects with |
| | | | from | Improvement from |
| | Mean ± S.D. | p-value | Baseline | Baseline |
| Baseline | 749.1 ± 241.5 | — | — | — |
| Week 1 | 661.0* ± 65.7 | <0.001 | −11.8% | 100% |
| Week 4 | 631.0* ± 76.8 | <0.001 | −15.8% | 97% |
| Week 8 | 588.6* ± 51.7 | <0.001 | −21.4% | 100% |

*Statistically significant difference from baseline, p < 0.05

When images taken after 1, 4 and 8 weeks of product were compared with baseline images, there were mean improvements of 11.8%, 15.8 and 21.4%, respectively, based on image analysis. The improvements observed were highly significant compared with baseline. A total of 100%, 97% and 100% of the subjects showed improvement after 1, 4 and 8 weeks of product use, respectively.

### Example 1.2: Corneometer® Measurements—Face

At baseline, immediately after the first use and after 1, 4 and 8 weeks of product use, a trained technician took Corneometer® measurements on the face of each subject to measure the moisture content of the skin. An increase in Corneometer® measurements indicates an improvement. Table 1.2 presents a summary of Corneometer® measurements.

TABLE 1.2
Corneometer ® Measurements

| | | | Mean | |
|---|---|---|---|---|
| | | | Change | % of Subjects with |
| | | | from | Improvement from |
| | Mean ± S.D. | p-value | Baseline | Baseline |
| Baseline | 28.8 ± 4.8 | — | — | — |
| Post-app. | 50.0* ± 9.6 | <0.001 | 73.6% | 100% |
| Week 1 | 62.8* ± 11.4 | <0.001 | 118% | 100% |
| Week 4 | 62.6* ± 13.6 | <0.001 | 117% | 100% |
| Week 8 | 55.5* ± 9.5 | <0.001 | 92.7% | 100% |

*Statistically significant difference from baseline, p < 0.05

When measurements taken immediately post-application and after 1, 4 and 8 weeks of product use were compared with baseline measurements, there were mean improvements of 73.6%, 118%, 117% and 92.7%, respectively, based on Corneometer® measurements. The improvements observed were highly significant compared with baseline. All of the subjects showed improvement immediately post-application and after 1, 4 and 8 weeks of product use.

### Example 1.3 Corneometer® Measurements—Arm

At baseline, immediately post-application and 24-, 48-, and 72-hours post-application, a trained technician took Corneometer® measurements on the volar arm of each subject to measure the moisture content of the skin. An increase in Corneometer® measurements indicates an improvement. Table 1.3 presents a summary of Corneometer® measurements.

TABLE 1.3
Corneometer ® Measurements

| | | | Mean | |
|---|---|---|---|---|
| | | | Change | % of Subjects with |
| | | | from | Improvement from |
| | Mean ± S.D. | p-value | Baseline | Baseline |
| Baseline | 28.5 ± 4.1 | — | — | — |
| Post-app. | 46.7* ± 8.6 | <0.001 | 63.9% | 100% |
| 24 Hours | 44.2* ± 9.7 | <0.001 | 55.1% | 100% |
| 48 Hours | 47.2* ± 115 | <0.001 | 65.6% | 97% |
| 72 Hours | 42.7* ± 8.9 | <0.001 | 49.8% | 100% |

*Statistically significant difference from baseline, p < 0.05

When measurements taken immediately post-application and after 24, 48 and 72 hours were compared with baseline measurements, there were mean improvements of 63.9%, 55.1%, 65.6% and 49.8%, respectively, based on Corneometer® measurements. The improvements observed were highly significant compared with baseline. A total of 100%, 100%, 97% and 100% of the subjects showed improvement immediately post-application and after 24, 48 and 72 hours, respectively.

### Example 1.4: Cutometer® RO Measurements

At baseline and after 1, 4 and 8 weeks of product use, a trained technician took Cutometer® measurements on each subject to measure the firmness of the skin. A decrease in the measurements indicates an improvement. Table 1.4 presents a summary of Cutometer® measurements.

TABLE 1.4
Cutometer ® RO Measurements

| | | | Mean | |
|---|---|---|---|---|
| | | | Change | % of Subjects with |
| | | | from | Improvement from |
| | Mean ± S.D. | p-value | Baseline | Baseline |
| Baseline | 0.191 ± 0.062 | — | — | — |
| Week 1 | 0.104* ± 0.042 | <0.001 | −45.5% | 82% |
| Week 4 | 0.094* ± 0.065 | <0.001 | −50.8% | 91% |
| Week 8 | 0.121* ± 0.024 | <0.001 | −36.6% | 79% |

*Statistically significant difference from baseline, p < 0.05

When measurements taken after 1, 4 and 8 weeks of use were compared with baseline measurements, there were mean improvements of 45.5%, 50.8% and 36.6%, respectively, based on Cutometer® RO measurements. The improvements observed after 1, 4 and 8 weeks of product use were highly significant compared with baseline. A total of 82%, 91% and 79% of the subjects showed improvement after 1, 4 and 8 weeks of use, respectively.

### Example 1.5: Cutometer® R2 Measurements

At baseline and after 1, 4 and 8 weeks of product use, a trained technician took Cutometer® measurements on each subject to measure the elasticity of the skin. An increase in the measurements indicates an improvement. Table 1.5 presents a summary of Cutometer® measurements.

TABLE 1.5
Cutometer ® R2 Measurements

| | | | Mean | |
|---|---|---|---|---|
| | | | Change | % of Subjects with |
| | | | from | Improvement from |
| | Mean ± S.D. | p-value | Baseline | Baseline |
| Baseline | 0.528 ± 0.075 | — | — | — |
| Week 1 | 0.636* ± 0.081 | <0.001 | 20.5% | 85% |
| Week 4 | 0.604* ± 0.104 | 0.005 | 14.4% | 64% |
| Week 8 | 0.663* ± 0.092 | <0.001 | 25.6% | 85% |

*Statistically significant difference from baseline, p < 0.05

When measurements taken after 1, 4 and 8 weeks of product use were compared with the baseline measurements, there were mean improvements of 20.5%, 14.4% and 25.6%, respectively based on Cutometer® measurements. The improvements observed after 1, 4 and 8 weeks of product use were statistically significant compared with baseline. A total of 85%, 64% and 85% of the subjects showed improvement after 1, 4 and 8 weeks of product use, respectively.

### Example 1.6: Tewameter® Measurements

At baseline, and after 1, 4 and 8 weeks of product use, a trained technician took Tewameter® measurements on the face of each subject to measure the skin barrier function. A decrease in Tewameter® measurements indicates an improvement. Table 2.6 presents a summary of Tewameter® measurements.

TABLE 1.6
Tewameter ® Measurements

| | | | Mean | |
|---|---|---|---|---|
| | | | Change | % of Subjects with |
| | | | from | Improvement from |
| | Mean ± S.D. | p-value | Baseline | Baseline |
| Baseline | 12.18 ± 2.16 | — | — | — |
| Week 1 | 10.08* ± 1.45 | <0.001 | −17.2% | 88% |
| Week 4 | 10.44* ± 1.59 | <0.001 | −14.3% | 76% |
| Week 8 | 8.97* ± 2.33 | <0.001 | −26.4% | 85% |

*Statistically significant difference from baseline, p < 0.05

When measurements taken after 1, 4 and 8 weeks of product use were compared with baseline measurements, there were mean improvements of 17.2%, 14.3% and 26.4%, respectively, based on Tewameter® measurements. The improvements observed after 1, 4 and 8 weeks of product use were highly significant compared with baseline. A total of 88%, 76% and 85% of the subjects showed improvement after 1, 4 and 8 weeks of product use, respectively.

### Example 1.7: Skin Irritation—Technician Evaluation

At each visit, a trained technician evaluated the face of each subject for irritation according to the scale below. This evaluation was for safety purposes only and was not used in determining efficacy.

Scale for Scoring Irritation
0=No irritation present
+=Barely perceptible irritation present
1=Mild irritation present
2=Moderate irritation present
3=Marked irritation present
4=Severe irritation present

TABLE 1.7
Skin Irritation-Technician Evaluation

| | | Mean | |
|---|---|---|---|
| | | Change | % of Subjects with |
| | | from | Improvement from |
| | Mean | Baseline | Baseline |
| Baseline | 0.0 | — | — |
| Post- | 0.0 | 0% | 0% |
| App. | | | |
| Week 1 | 0.0 | 0% | 0% |
| Week 4 | 0.0 | 0% | 0% |
| Week 8 | 0.0 | 0% | 0% |

There was no irritation observed on any subject during the course of the study.

Conclusions

Global fine lines and wrinkles were significantly improved after 1, 4 and 8 weeks of use, based on image analysis.

Skin moisture on the face and the arm was significantly improved immediately after the first use, 24, 48 and 72 hours after the first use and after 1, 4 and 8 weeks of use, based on Corneometer measurements.

Skin barrier function was significantly improved after 1, 4 and 8 weeks of use, based on Tewameter measurements.

In just one night, 100% had visibly plumper skin with lines and wrinkles visible reduced. In four weeks 91% had visible firmer skin, and 100% continued to show a visible reduction in wrinkles.

### Example 2: In Vitro Combination Treatment Study

The purpose of this study was to evaluate the hyaluronic acid (HA) expression change activity of RoC® HA Serum Capsules (i.e. Example 2.0), RoC® Retinol Serum Capsules (i.e. Example 1.0), commercially available HA Serum (1.5 wt. % HA) and commercially available Retinol Serum (0.3 wt. % retinol) compared to 0.15% Retinol-treated tissues in cultured human epidermal/dermal skin tissues (EpiDerm-FT™). HA levels from culture medium, tissue lysates, HA histology and gene expression related to dermal anti-aging effects were quantified using qPCR method after 24- and 72-hours incubation with test materials.

### Example 2.1—Skin Hydration

After incubations, tissues were fixed and subjected to the normal steps of tissue preparation for histological analysis: dehydration, paraffin embedding, sectioning and staining with Alcian Blue. Histological activity was analyzed using image analysis including blue-color deconvolution and luminosity per section area in dermal layer. The percent change effect of each treatment was calculated comparing the vehicle-only treated tissues group. Results produced by image analysis densitometry changes for each treatment showed that all test materials produced different spectrum in HA staining activity.

TABLE 2.1
Hyaluronic acid levels and Alcian blue staining change (%) results after test material
treatments in EpiDerm-FT

| | Hyaluronic Acid (HA) | | | | Histology | |
|---|---|---|---|---|---|---|
| | Culture medium | | Tissue lysates | | Alcian Blue in Dermis | |
| Treatment | 24 hr | 72 hr | 24 hr | 72 hr | 24 hr | 72 hr |
| 0.15% Retinol in DMSO | +34 | +25 | +52 | +49 | +81 | −9 |
| RoC ® HA (0.006 wt. %) Serum Capsules | +66 | +52 | +97 | +39 | +83 | 0 |
| RoC ® Retinol (0.22 wt. %) Serum Capsules | +81 | +49 | +61 | +33 | +143 | +36 |
| RoC ® HA Capsules + 0.15% Retinol | +46 | +38 | +56 | +84 | +121 | −27 |
| RoC ® HA Capsules + RoC ® Retinol | +12 | +10 | 24 | +13 | +111 | +1 |
| Commercially available ® HA (1.5 wt. %) | +48 | +1 | +44 | +43 | +26 | +37 |
| Commercially available Retinol (0.3 wt. %) | +19 | −21 | 6 | −4 | +124 | +4 |

HA was extracted after 24- and 72-hours treatments. Data indicates average data (n = 3 tissues per test group) levels change percentages were calculated based on HA ELISA analysis using vehicle-treated tissues as reference. Bold = Significant data compared to vehicle control group (p < 0.05). ‘+’ indicates levels increase %. ‘−’ indicates levels decrease %.

Hyaluronic Acid (HA) levels results produced by ELISA method showed that 0.15% Retinol formulated in DMSO significantly increased HA levels in both culture medium (34%) and tissue lysates (52%) after 24 hours. RoC® Retinol capsules significantly increased HA levels in culture medium (81%) and tissue lysates (61%) after 24 hours and in culture medium (49%) after 72 hours. Commercially available Retinol serum only significantly increased HA in culture medium (19%) after 24 hours. Conversely, RoC® HA capsules significantly increased HA levels in culture medium (66%) and tissue lysates (97%) after 24 hours and in culture medium (52%) after 72 hours. Commercially available HA serum only significantly increased HA levels in both culture medium (48%) and tissue lysates (44%) after 24 hours. When RoC® HA capsules are blended in combination with 0.15% Retinol, this treatment significantly increased HA levels in tissue lysates by 84% after 72 hours better than their individual treatments. In addition, when RoC® HA capsules are blended in combination with RoC® Retinol capsules, this treatment did not significantly increase HA levels better than their individual treatments.

RoC® Retinol capsules and Commercially available Retinol serum products significantly increased staining intensity in dermal layer after 24 hours by 143% and 124%, respectively. In addition, when RoC® HA capsules are blended in combination with 0.15% Retinol or RoC® Retinol capsules, both treatments significantly increased HA levels in tissue lysates by 121% and 111%, respectively. In contrast, no significant changes were obtained after 72 hours treatments.

### Example 2.2—Gene Expression

Gene expression produced by RT-PCR method showed that 0.15% Retinol formulated in DMSO significantly increased Fibronectin (FN1, 45%) gene after 72 hours. RoC® Retinol capsules significantly increased Hyaluronic acid synthase-2 (HAS2, 125%) and FN1 (30%) after 24 hours. Commercially available Retinol serum significantly increased HAS2 (233%) after 24 hours and Elastin (ELN, 40%) after 72 hours. Conversely, RoC® HA capsules significantly increased Hyaluronic acid synthase-1 (HAS1, 463%) after 24 hours and ELN (78%) after 72 hours. Commercially available HA serum significantly increased HAS2 (298%) after 24 hours and ELN (37%) after 72 hours. Interestingly, when RoC® HA capsules are blended in combination with 0.15% Retinol, this treatment significantly increased Collagen type-I (COL1A1, 48%), Collagen type-III (COL3A1, 58%), Collagen type-IV (COL4A1, 79%), ELN (52%) and FN1 (108%) after 72 hours better than their individual treatments for these genes. In addition, when RoC® HA capsules are blended in combination with RoC® Retinol capsules, this treatment significantly increased HAS2 (638%), COL3A1 (58%) after 24 hours and FN1 (59%) after 72 hours, again better than their individual treatments for these genes.

TABLE 2.2
Gene Expression change (%) results after test material treatments in EpiDerm-FT ™

| | Skin-ECM Structure | | | | | | | | | | Skin Hydration | | | |
|---|---|---|---|---|---|---|---|---|---|---|---|---|---|---|
| | COL1A1 | | COL3A1 | | COL4A1 | | ELN | | FN1 | | HAS1 | | HAS2 | |
| Treatment | 24 hr | 72 hr | 24 hr | 72 hr | 24 hr | 72 hr | 24 hr | 72 hr | 24 hr | 72 hr | 24 hr | 72 hr | 24 hr | 72 hr |
| 0.15% | −22 | +18 | −22 | +8 | −30 | +9 | −21 | +25 | −16 | +45 | −52 | −29 | −11 | −18 |
| Retinol | | | | | | | | | | | | | | |
| RoC ® HA | +18 | +12 | +18 | +8 | +9 | +19 | −9 | +40 | +47 | +78 | −53 | +463 | +27 | +24 |
| (0.006 wt. %) | | | | | | | | | | | | | | |
| RoC ® Retinol | +16 | +22 | +2 | +1 | +20 | −27 | +26 | +20 | +30 | +12 | −9 | +55 | +125 | −18 |
| Capsules | | | | | | | | | | | | | | |
| (0.22 wt. %) | | | | | | | | | | | | | | |
| RoC ® HA | −4 | +48 | −10 | +58 | −47 | +79 | −25 | +52 | −24 | +108 | −71 | +2 | +5 | +33 |
| Capsules + | | | | | | | | | | | | | | |
| 0.15% | | | | | | | | | | | | | | |
| Retinol | | | | | | | | | | | | | | |
| RoC ® HA | +57 | +27 | +58 | +33 | −72 | +12 | −28 | +40 | −25 | +59 | −94 | +1 | +638 | +23 |
| Capsules + | | | | | | | | | | | | | | |
| RoC ® Retinol | | | | | | | | | | | | | | |
| Capsules | | | | | | | | | | | | | | |
| Commercially | +18 | +18 | +18 | +16 | −79 | −14 | −66 | +37 | −37 | +19 | −53 | +41 | +298 | +7 |
| available | | | | | | | | | | | | | | |
| HA (1.5 wt. %) | | | | | | | | | | | | | | |
| Serum | | | | | | | | | | | | | | |
| Commercially | +17 | +8 | +12 | +11 | −65 | −36 | −66 | +40 | −6 | −1 | −74 | −53 | +233 | −28 |
| available | | | | | | | | | | | | | | |
| Retinol (0.3 | | | | | | | | | | | | | | |
| wt. %) | | | | | | | | | | | | | | |
| Serum | | | | | | | | | | | | | | |

RNA extracted after 24- and 72-hours treatments. Data indicates average data (n = 3 tissues per test group) expression change percentages were calculated based on ^{2-ΔΔCt} analysis using vehicle-treated tissues as reference and GAPDH as housekeeping gene. Bold = Significant data compared to vehicle control group (p < 0.05). ‘+’ indicates gene expression increase %. ‘−’ indicates gene expression decrease %.

Conclusions

Based on this data, both RoC® Retinol and HA capsules produced the best response in HA production in cultured skin tissue models (EpiDerm-FT™). In addition, RoC® Retinol and HA capsules produced the best response in Fibronectin and Hyaluronic acid synthase-1 gene increase. Commercially available HA and retinol serum produced the best response in Elastin and Hyaluronic acid synthase-2 gene increase. The combination of RoC® HA capsules with 0.15% Retinol or RoC® Retinol capsules outperformed the gene expression increase in collagen, Elastin and Hyaluronic acid synthase-2 when compared to the individual treatments

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

Further aspects are provided by the subject matter of the following clauses.

A cosmetic composition including a hyaluronic acid (HA) compound, wherein the composition is anhydrous.

The cosmetic composition of the preceding clause, wherein the HA compound is free hyaluronic acid or a salt thereof.

The cosmetic composition of any preceding clause, wherein the composition further includes konjac glucomannan.

The cosmetic composition of any preceding clause, wherein the HA compound is crosslinked with the konjac glucomannan.

The cosmetic composition of any preceding clause, wherein the composition further includes a retinoid.

The cosmetic composition of any preceding clause, wherein the composition includes about 0.001 wt % to about 5.000 wt % of the retinoid.

The cosmetic composition of any preceding clause, wherein the composition includes about 0.1 wt % to about 0.5 wt. % of the retinoid.

The cosmetic composition of any preceding clause, wherein the retinoid is selected from retinol, retinal, tretinoin, isotretinoin, alitretinoin, etretinate acitretin, adapalene, bexarotene, and tazarotene, and trifarotene.

The cosmetic composition of any preceding clause, wherein the retinoid is retinol.

The cosmetic composition of any preceding clause, wherein the composition includes about 0.001 wt. % to about 3.000 wt. % of the HA compound.

The cosmetic composition any one of any preceding clause, wherein the composition includes about 0.006 wt. % of the HA compound.

The cosmetic composition of any preceding clause, wherein the HA has a molecular weight of about 1 kDa to about 10000 kDa.

The cosmetic composition of any preceding clause, wherein the HA compound includes a HA having a molecular weight of about 10 kDa to about 40 kDa.

The cosmetic composition of any preceding clause, wherein the HA compound includes a HA having a molecular weight of about 25 kDa to 100 kDa.

The cosmetic composition of any preceding clause, wherein the HA compound includes a HA having a molecular weight of about 1 kDa to about 20 kDa.

A method of improving skin including topically administering a hyaluronic acid (HA) compound and a retinoid in one or more compositions to the skin of a subject, wherein improving the skin includes (a) increasing one or more of collagen, elastin, fibronectin, and hyaluronic acid gene expression in the skin of a subject and/or (b) improving the look, feel and/or appearance of the skin, by hydrating the skin; plumping the skin; improving skin elasticity; improving the appearance of fine lines and wrinkles; reducing the depth, length, or width of fine lines and wrinkles; and/or smoothing the skin; and/or (c) increasing and/or replenishes the level of or increases production of hyaluronic acid on the surface of the skin and/or in the skin.

The method of the preceding clause, wherein the HA compound is hyaluronic acid or a salt thereof.

The method of any preceding clause, wherein the retinoid is selected from retinol, retinal, tretinoin, isotretinoin, alitretinoin, etretinate, acitretin, adapalene, bexarotene, tazarotene, and trifarotene.

The method of any preceding clause, wherein the retinoid is retinol.

The method of any preceding clause, further including administering konjac glucomannan.

The method of any preceding clause, wherein the HA compound is crosslinked with the konjac glucomannan.

The method of any preceding clause, wherein the one or more compositions are anhydrous.

The method of any preceding clause, wherein the method improves fine lines and wrinkles of the skin, increases skin firmness, increases skin plumpness, increases smoothness of the skin, and/or increases elasticity of the skin.

The method of any preceding clause, wherein the method increases hydration in the skin and/or increases the levels of hyaluronic acid on the surface of the skin and/or in the skin.

The method of any preceding clause, wherein the collagen, elastin, fibronectin, and hyaluronic acid gene expression is selected from one or more of hyaluronic acid synthase 1 (HAS1), hyaluronic acid synthase 2 (HAS2), collagen type-I (COL1A1), collagen type-III (COL3A1), and collagen type-IV (COL4A1), elastin (ELN), and fibronectin (FN1) gene expression.

The method of any preceding clause, wherein the HA compound and the retinoid are administered in separate compositions.

The method of any preceding clause, wherein the HA compound and the retinoid are administered in the same composition.

The method of any preceding clause, wherein one of the one or more compositions includes about 0.001 wt % to about 5.000 wt % of the retinoid.

The method of any preceding clause, wherein one of the one or more compositions includes about 0.1 wt. % to about 0.5 wt. % of the retinoid.

The method of any preceding clause, wherein the one of the one or more compositions includes about 0.2 wt % of the retinoid.

The method of any preceding clause, wherein one of the one or more compositions includes about 0.001 wt. % to about 3.000 wt. % of the HA compound.

The method of any preceding clause, wherein one of the one or more compositions includes about 0.006 wt. % of the HA compound.

The method of any preceding clause, wherein the HA compound has a molecular weight of about 1 kDa to about 10000 kDa.

The method of any preceding clause, wherein the HA compound includes a HA compound having a molecular weight of about 25 kDa to 100 kDa.

The method of any preceding clause, wherein the HA compound includes a HA compound having a molecular weight of about 1 kDa to about 20 kDa.

The method of any preceding clause, wherein the HA compound includes a HA compound having a molecular weight of less than or equal to about 40 kDa.

The method of any preceding clause, wherein the HA compound includes a HA compound having a molecular weight about 10 kDa to about 40 kDa.

The method of any preceding clause, wherein HA compound is administered prior to the retinoid.

The method of any preceding clause, wherein the HA compound and the retinoid are administered simultaneously.

The method of any preceding clause, wherein the one or more compositions is administered in the evening.

The method of any preceding clause, wherein a first of the one or more compositions is administered in the morning and a second of the one or more compositions is administered in the evening.

The method of any preceding clause, wherein the one or more compositions is administered daily.

The method of any preceding clause, wherein the skin is the skin of the face, of the neck, or of both the face and the neck.

The method of any preceding clause, wherein the skin is cleansed prior to administration of the hyaluronic acid and the retinoid.

A method of improving skin including administering the composition of any preceding clause to improve the look, feel and/or appearance of the skin.

A method of improving skin including topically administering to the skin hyaluronic acid and a retinoid to improve the look, feel and/or appearance of the skin.

The method of any preceding clause, wherein improving the look, feel and/or appearance of the skin, includes one or more of hydrating the skin, plumping the skin, improving skin elasticity, improving the appearance of fine lines and wrinkles, reducing the depth, length, or width of fine lines and wrinkles, and smoothing the skin.

A method of improving skin including topically administering a hyaluronic acid (HA) compound in a composition to the skin of a subject, wherein improving the skin includes (a) increasing one or more of collagen, elastin, fibronectin, and hyaluronic acid gene expression in the skin of a subject and/or (b) improving the look, feel and/or appearance of the skin, by hydrating the skin; plumping the skin; improving skin elasticity; improving the appearance of fine lines and wrinkles; reducing the depth, length, or width of fine lines and wrinkles; and/or smoothing the skin; and/or (c) increasing and/or replenishes the level or increasing production of hyaluronic acid, on the surface of the skin and/or in the skin.

The method of the preceding clause, wherein the composition is a cream, gel, lotion, emulsion, or serum.

The method of any preceding clause, wherein the composition is anhydrous.

The method of any preceding clause, wherein the HA compound has a molecular weight of about 1 kDa to about 10000 kDa.

The method of any preceding clause, wherein the HA compound includes a HA compound having a molecular weight of about 25 kDa to 100 kDa.

The method of any preceding clause, wherein the HA compound includes a HA compound having a molecular weight of about 1 kDa to about 20 kDa.

The method of any preceding clause, wherein the HA compound includes a HA compound having a molecular weight of less than or equal to about 40 kDa.

The method of any preceding clause, wherein the HA compound includes a HA compound having a molecular weight about 10 kDa to about 40 kDa.

The method of any preceding clause, wherein the composition is a composition as of any preceding clause.

The method of any preceding clause, further including administering a retinoid, wherein the HA compound and the retinoid are in one or more compositions.

1. A cosmetic composition comprising a hyaluronic acid (HA) compound, wherein the composition is anhydrous.

2. The cosmetic composition of claim 1, wherein the HA compound is free HA or a salt thereof.

3. The cosmetic composition of claim 1, wherein the composition further comprises konjac glucomannan.

4. The cosmetic composition of claim 3, wherein the HA compound is crosslinked with the konjac glucomannan.

5. The cosmetic composition of claim 1, wherein the composition further comprises a retinoid.

6. The cosmetic composition of claim 5, wherein the composition comprises about 0.001 wt % to about 5.000 wt % of the retinoid.

7. (canceled)

8. The cosmetic composition of claim 5, wherein the retinoid is selected from the group consisting of retinol, retinal, tretinoin, isotretinoin, alitretinoin, etretinate acitretin, adapalene, bexarotene, and tazarotene, and trifarotene.

9. (canceled)

10. The cosmetic composition of claim 1, wherein the composition comprises about 0.001 wt. % to about 3.000 wt. % of the HA compound.

11-12. (canceled)

13. The cosmetic composition of claim 1, wherein the HA compound comprises a HA having a molecular weight of about 10 kDa to about 40 kDa.

14-24. (canceled)

25. The method of claim 48, wherein the collagen, elastin, fibronectin, and hyaluronic acid gene expression is selected from one or more of hyaluronic acid synthase 1 (HAS1), hyaluronic acid synthase 2 (HAS2), collagen type-I (COL1A1), collagen type-III (COL3A1), and collagen type-IV (COL4A1), elastin (ELN), and fibronectin (FN1) gene expression.

26-47. (canceled)

48. A method of improving skin comprising topically administering a hyaluronic acid (HA) compound in a composition to the skin of a subject, wherein improving the skin comprises (a) increases one or more of collagen, elastin, fibronectin, and hyaluronic acid gene expression in the skin of a subject and/or (b) improves the look, feel and/or appearance of the skin, by hydrating the skin; plumping the skin; improving skin elasticity; improving the appearance of fine lines and wrinkles; reducing the depth, length, or width of fine lines and wrinkles; and/or smoothing the skin; and/or (c) increases and/or replenishes the level or increases production of hyaluronic acid, on the surface of the skin and/or in the skin.

49. The method of claim 48, wherein the composition is a cream, gel, lotion, emulsion, or serum.

50. The method of claim 48, wherein the composition is anhydrous.

51. The method of claim 48, wherein the HA compound has a molecular weight of about 1 kDa to about 10000 kDa.

52. The method of claim 51, wherein the HA compound comprises a HA compound having a molecular weight of about 25 kDa to 100 kDa.

53. The method of claim 51, wherein the HA compound comprises a HA compound having a molecular weight of about 1 kDa to about 20 kDa.

54. The method of claim 51, wherein the HA compound comprises a HA compound having a molecular weight of less than or equal to about 40 kDa.

55. The method of claim 51, wherein the HA compound comprises a HA compound having a molecular weight about 10 kDa to about 40 kDa.

56. The method of claim 48, further comprising administering a retinoid, wherein the HA compound and the retinoid are in one or more compositions.

57. The method of claim 48, wherein the composition is the composition of claim 1.
